(19) United States
(12) Patent Application Publication
(10) Pub. No.: US 2026/0151459 A1
(43) Pub. Date: Jun. 4, 2026

(54) Composition of GLP-1 and PYY3-36 Polypeptide Analogues, and Preparation Method and Use Thereof

(71) Applicant: Tao Sun, Xiamen (CN)

(72) Inventor: Tao Sun, Xiamen (CN)

(21) Appl. No.: 18/999,152

(22) Filed: Dec. 23, 2024

(30) Foreign Application Priority Data
Nov. 29, 2024 (CN) 202411746099.X

(51) Int. Cl. A61K 38/26; A61K 9/00; A61K 9/20; A61K 9/48; A61P 1/16; A61P 3/04; A61P 3/10; A61P 25/22; A61P 25/24

(52) U.S. Cl. CPC A61K 38/26; A61K 9/0019; A61K 9/2009; A61K 9/2018; A61K 9/2054; A61K 9/2059; A61K 9/4825; A61K 9/4858; A61K 9/4866; A61P 1/16; A61P 3/04; A61P 3/10; A61P 25/22; A61P 25/24

(57) ABSTRACT

The present disclosure discloses a composition of GLP-1 and PYY₃₋₃₆ polypeptide analogues, a preparation method and use thereof, and relates to the technical field of biomedicines. The composition includes a GLP-1 polypeptide analogue and a PYY₃₋₃₆ polypeptide analogue. Compared with the natural sequence of GLP-1 and the natural sequence of PYY₃₋₃₆, the composition of the GLP-1 polypeptide analogue and the PYY₃₋₃₆ polypeptide analogue has remarkable effects in ameliorating metabolic diseases such as obesity, diabetes mellitus, and fatty liver, and mood disorders such as anxiety and depression.

### CROSS-REFERENCE TO RELATED APPLICATION

This application claims priority to Chinese Patent Application No. 202411746099.X, filed Nov. 29, 2024, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2409012.xml. The size of the file is 9,511 bytes, and the file was created on Dec. 19, 2024.

### BACKGROUND

### Technical Field

The present disclosure relates to the technical field of biomedicines, in particular to a composition of GLP-1 and PYY₃₋₃₆ polypeptide analogues, a preparation method and use thereof.

### Technical Considerations

Obesity increases the risk of diabetes mellitus (DM), fatty liver (FL), and other diseases. The World Health Organization defines obesity as a chronic disease that develops from excessive or abnormal adipose tissues caused by the disorder of normal body fat mass regulation. The development and severity are influenced by endogenous and exogenous (environmental) factors ^{[1, 2]}. DM is a severe chronic metabolic disorder, which is caused by the interaction of genetic and environmental factors and manifests as insufficient insulin secretion from islet β cells and insulin resistance in tissues, which leads to increased blood glucose. Sedentary, lack of exercise, smoking, and alcohol drinking are the main risk factors for DM ^{[3, 4]}. FL is a disease characterized by pathological changes in the liver. FL occurs when lipid accumulation in hepatocytes exceeds 5% of the wet weight of the liver. Unhealthy diet and lack of physical activity are determining factors for FL ^{[5, 6]}.

Human glucagon-like peptide-1 (GLP-1) is a polypeptide composed of 31 amino acids. After binding with its receptor, GLP-1 exerts biological effects, including suppressing appetite, delaying gastric emptying, and inducing body weight loss, and can regulate inflammation in many parts of the body and ameliorate diseases related to the metabolic system, cardiovascular system, nervous system, digestive system, etc. ^{[7, 8]}.

Human peptide tyrosine tyrosine (PYY) is a polypeptide composed of 36 amino acids. PYY₃₋₃₆ is the main existing form of PYY in the body. After binding with the receptor, it exerts biological effects, including inhibiting gastrointestinal peristalsis and pancreatic secretion, changing intestinal permeability, increasing the absorption of water and electrolytes in the colon, etc., and can regulate inflammation in many parts of the body and ameliorate diseases related to the metabolic system, cardiovascular system, nervous system, digestive system, etc. ^{[9, 10]}.

In recent years, combined administration of multiple endogenous peptides or proteins with complementary effects has become a new concept in pharmacological intervention. For example, the use of a glucagon-like peptide-1 receptor agonist and a sodium-glucose cotransporter inhibitor in combination more effectively treats diabetes mellitus, and the use of PYY₃₋₃₆ and islet amyloid polypeptide in combination more effectively reduces the body weight ^{[11, 12]}.

### SUMMARY

The present disclosure provides a composition, which has remarkable effects in ameliorating diseases associated with GLP-1 and/or PYY3-36, such as obesity, DM, and FL, as well as anxiety, depression, etc.

The present disclosure further provides the use of the composition in preparing a drug.

The present disclosure further provides a method for preventing and/or treating a disease.

The present disclosure further provides a drug comprising the above composition.

The present disclosure further provides the use of the composition in preparing a health care product or a nutritional supplement or as a food additive ingredient.

The first aspect of the present disclosure relates to a composition comprising a GLP-1 polypeptide analogue and a PYY₃₋₃₆ polypeptide analogue, wherein the amino acid sequences of the GLP-1 polypeptide analogue are as shown in Formula I, and the amino acid sequences of the PYY₃₋₃₆ polypeptide analogue are as shown in Formula II:

(SEQ ID NO: 3)
HX₂ₐX₃ₐ GTF TSD VSS YLE GQA AKX₂₁ₐ FIA WLX₂₇ₐ KX₂₉ₐR X₃₁ₐ
Formula I

X₂ₐ is selected from the group consisting of valine, isoleucine, glycine, threonine, leucine, and serine;
X₃ₐ is selected from the group consisting of serine and alanine;
X₂₁ₐ is selected from the group consisting of arginine and lysine;
X₂₇ₐ is selected from the group consisting of serine and alanine;
X₂₉ₐ is selected from the group consisting of alanine, serine, threonine, and methionine; and
X₃₁ₐ is selected from the group consisting of alanine, serine, threonine, and methionine; and
wherein the amino acid sequences of the PYY₃₋₃₆ polypeptide analogue are as shown in Formula II:

(SEQ ID NO: 4)
IKP EAP GEX_{11b} ASP EEL X_{18b}X_{19b}Y YAX_{23b} LRH X_{27b}LN X_{30b}VT RQR Y
Formula II

X_{11b} is selected from the group consisting of aspartic acid, glycine, glutamic acid, and asparagine;
X_{18b} is selected from the group consisting of aspartic acid, glutamine, and histidine;
X_{19b} is selected from the group consisting of arginine and lysine;
X_{23b} is selected from the group consisting of serine, threonine, alanine, glycine, and methionine;
X_{27b} is selected from the group consisting of tyrosine, tryptophan, and phenylalanine; and
X_{30b} is selected from the group consisting of leucine, alanine, isoleucine, valine, and methionine.

According to some non-limiting embodiments of the present disclosure, the amino acid sequences of the GLP-1 polypeptide analogue are as shown in Formula III:

(SEQ ID NO: 1)
HGE GTF TSD VSS YLE GQA AKE FIA WLV KSRA
Formula III.

According to some non-limiting embodiments of the present disclosure, the amino acid sequences of the PYY₃₋₃₆ polypeptide analogue are as shown in Formula IV:

(SEQ ID NO: 2)
IKP EAP GED ASP EEL VRY YAG LRH WLN LVT RQRY
Formula IV.

According to some non-limiting embodiments of the present disclosure, the mass ratio of the GLP-1 polypeptide analogue to the PYY₃₋₃₆ polypeptide analogue is 1:0.25-4. For example, the mass ratio of the GLP-1 polypeptide analogue to the PYY₃₋₃₆ polypeptide analogue may be 1:0.25, 1:0.5, 1:0.75, 1:1, 1:1.25, 1:1.5, 1:1.75, 1:2, 1:2.25, 1:2.5, 1:2.75, 1:3, 1:3.25, 1:3.5, 1:3.75, or 1:4.

The second aspect of the present disclosure relates to the use of the composition for treating a disease, or the use of the composition in preparing a drug for preventing and/or treating a disease.

According to some non-limiting embodiments of the present disclosure, diseases that the drug is used to prevent and/or treat include but are not limited to obesity and related complications, diabetes mellitus and related complications, glucose intolerance and related diseases caused by glucose intolerance, hyperglycemia, hyperinsulinemia, hypertension, dyslipidemia, cognitive disorder, atherosclerosis, myocardial infarction, cardiovascular disease, stroke, related diseases caused by intestinal permeability disorder, such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, nephropathy, asthma, chronic obstructive pulmonary disease, dysmetabolic syndrome, mood disorder, neurodegenerative disease, systemic lupus erythematosus, etc.

According to some non-limiting embodiments of the present disclosure, the mood disorder includes at least one of anxiety and depression.

According to some non-limiting embodiments of the present disclosure, the neurodegenerative disease includes Alzheimer's disease.

According to some non-limiting embodiments of the present disclosure, the drug has at least one of functions B1) to B4):
B1) reducing food intake;
B2) reducing body weight;
B3) increasing insulin sensitivity; and
B4) inhibiting fat accumulation.

According to some non-limiting embodiments of the present disclosure, the fat accumulation in B4) includes at least one of subcutaneous fat accumulation, inter-organ fat accumulation, and liver fat accumulation.

The third aspect of the present disclosure relates to a method for preventing and/or treating a disease, comprising administering a therapeutically effective amount of the above composition to a subject in need thereof.

According to some non-limiting embodiments of the present disclosure, the disease includes at least one of obesity and related complications, diabetes mellitus and related complications, glucose intolerance and related diseases caused by glucose intolerance, hyperglycemia, hyperinsulinemia, hypertension, dyslipidemia, cognitive disorder, atherosclerosis, myocardial infarction, cardiovascular disease, stroke, related diseases caused by intestinal permeability disorder, such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, nephropathy, asthma, chronic obstructive pulmonary disease, dysmetabolic syndrome, mood disorder, neurodegenerative disease, and systemic lupus erythematosus.

According to some non-limiting embodiments of the present disclosure, the mood disorder includes at least one of anxiety and depression.

According to some non-limiting embodiments of the present disclosure, the neurodegenerative disease includes Alzheimer's disease.

According to some non-limiting embodiments of the present disclosure, the mass ratio of the dosage of the GLP-1 polypeptide analogue to that of the PYY₃₋₃₆ polypeptide analogue is 1:0.25-4.

The fourth aspect of the present disclosure relates to a drug comprising the above composition and an optional pharmaceutically acceptable excipient.

According to some non-limiting embodiments of the present disclosure, the dosage form of the drug is selected from tablets, capsules, an injection, etc. The selected dosage form is suitable for the route of administration.

According to some non-limiting embodiments of the present disclosure, the excipient includes at least one of a filler, a binder, a lubricant, a solvent, a disintegrant, a buffer, and a preservative.

The fifth aspect of the present disclosure relates to the use of the above composition in preparing a health care product or a nutritional supplement or as a food additive ingredient.

The present disclosure has at least the following beneficial effects.

The composition comprises a GLP-1 polypeptide analogue and a PYY₃₋₃₆ polypeptide analogue. Compared with the natural sequence of GLP-1 and the natural sequence of PYY₃₋₃₆, the composition of the GLP-1 polypeptide analogue and the PYY₃₋₃₆ polypeptide analogue has remarkable effects in ameliorating metabolic diseases such as obesity, diabetes mellitus, and fatty liver, and mood disorders such as anxiety and depression.

The other features and advantages of the present disclosure will be set forth in the description which follows, and in part will be obvious from the description, or may be learned by the practice of the present disclosure.

### BRIEF DESCRIPTION OF THE DRAWINGS

FIG. 1 illustrates the effect of Formula III (denoted by G1, the same below) and Formula IV (denoted by P1, the same below) administered in combination on the cytotoxicity of HEK-293t in a CCK-8 detection test;

FIG. 2 illustrates a grouping diagram of mouse experiments in Experimental Example 3;

FIGS. 3a and 3b illustrate the effects of Formula III, Formula IV, and a combination thereof administered in combination (denoted by G1P1, the same below) on the body weight and food intake of HFD mice, wherein FIG. 3a illustrates the body weight change rate; and FIG. 3b illustrates the food intake change rate;

FIGS. 4a-4d illustrate the effects of Formula III, Formula IV, and a combination thereof administered in combination on anxiety and depression symptoms in mice, wherein FIG. 4a and

FIG. 4b respectively show the residence time in a central area in an open field test and the number of times of access to the central area, FIG. 4c shows the residence time in an open arm area in an elevated plus maze test, and FIG. 4d shows the time of immobility in a tail suspension test.

FIG. 5 illustrates the effects of Formula III, Formula IV, and a combination thereof administered in combination on blood glucose of HFD mice in a GTT test;

FIG. 6 illustrates the effects of Formula III, Formula IV, and a combination thereof administered in combination on blood glucose of HFD mice in an ITT test;

FIGS. 7a and 7b illustrate the effects of Formula III, Formula IV, and a combination thereof administered in combination on fatty liver in an oil red O staining test, wherein FIG. 7a shows oil red O staining in liver, and FIG. 7b shows the quantification of oil red O staining in liver;

FIGS. 8a and 8b illustrate the effects of Formula III, Formula IV, and a combination thereof administered in combination on muscle in an HE staining test, wherein FIG. 8a shows HE staining in muscle, and FIG. 8b shows HE quantification in muscle; and

FIGS. 9a and 9b illustrate the effects of Formula III, Formula IV, and a combination thereof administered in combination on body weight in a research-oriented clinical trial, wherein FIG. 9a shows the body weight change, and FIG. 9b shows the body weight change rate.

### DETAILED DESCRIPTION

The following description sets forth exemplary embodiments of the present disclosure. It should be recognized that such a description is not intended as a limitation on the scope of the present disclosure but is provided as a description of exemplary embodiments. Based on the examples of the present disclosure, other examples obtained by those skilled in the art without involving any inventive effort all fall within the scope of protection of the present disclosure.

Examples, in which no specific conditions are indicated, are carried out according to conventional conditions or conditions suggested by the manufacturer. The reagents or instruments used in which no manufacturer is indicated are all conventional products that can be purchased on the market.

When a numerical range is disclosed herein, the above range is regarded as continuous and includes the minimum and maximum values of the range, and every value between the minimum and maximum values. Furthermore, when a range refers to integers, it includes every integer between the minimum and maximum values of the range. Furthermore, when multiple ranges are provided to describe features or characteristics, the ranges can be combined. In other words, unless otherwise indicated, all ranges disclosed herein should be understood to include any and all subranges subsumed therein.

The term “and/or” is used to indicate that one or both of the stated situations may occur. For example, A and/or B include (A and B) and (A or B).

Unless otherwise indicated, the quantities of different ingredients and reaction conditions used herein can be interpreted as “approximate” and “about” in any case. Accordingly, unless otherwise specified, the numerical parameters stated in the following and claims are all rough parameters, and different numerical parameters may be obtained due to different standard errors under respective experimental conditions.

As used herein, the term “GLP-1 analogue” refers to a GLP-1 analogue or a variant thereof and is used to refer to a polypeptide of Formula I or III. The “GLP-1 analogue” has GLP-1 activity. Having GLP-1 activity means the ability to bind GLP-1 receptor and trigger a signal transduction pathway to produce an insulin-promoting effect or other physiological effects.

As used herein, the term “PYY₃₋₃₆ analogue” refers to a PYY analogue or a variant thereof and is used to refer to a polypeptide of Formula II or IV. The “PYY₃₋₃₆ analogue” has PYY₃₋₃₆ activity. Having PYY₃₋₃₆ activity means the ability to bind PYY₃₋₃₆ receptor and trigger signal transduction pathway to produce insulin-promoting effect or other physiological effects.

The GLP-1 analogue and PYY₃₋₃₆ analogue of the present disclosure can be prepared by using various polypeptide synthesis techniques well known to those skilled in the art.^{[13-15]}. The instruments and equipment needed for synthesis and the related reagents can all be purchased from conventional commercial channels. The GLP-1 analogue and PYY₃₋₃₆ analogue of the present disclosure can be synthesized in steps from the C-terminal of the peptide on an insoluble polymer carrier (also known as “resin”). The synthesis began by attaching the C-terminal amino acid of the peptide to the resin by forming an amide or ester bond. This led to the final release of the obtained peptide in the form of a C-terminal amide or carboxylic acid, respectively. Alternatively, if a C-terminal amino acid exists, the C-terminal residue as described herein may be attached to a 2-methoxy-4-alkoxybenzyl alcohol resin, and after the peptide sequence is assembled, the resulting peptide is released using a solution of lithium borohydride in tetrahydrofuran ^{[16]}.

It is known to those skilled in the art that amino acids exist in two isomers, D and/or L. The present disclosure comprises a mixture of one isomer and/or another isomer of amino acids, which is used for the synthesis of the peptide described herein.

The GLP-1 analogue and PYY₃₋₃₆ analogue of the present disclosure may also be produced by a genetic recombination method, i.e., by culturing a host cell containing a deoxyribonucleic acid sequence encoding the analogue and being capable of expressing the peptide in a suitable nutrient medium under conditions that allow for the expression of the peptide. Non-limiting examples of host cells suitable for expressing such peptides are Escherichia coli, Saccharomyce scerevisiae, and a mammalian baby hamster kidney cell line or Chinese hamster ovary cell line. In some non-limiting embodiments, this complete recombination fermentation step of the production process is satisfactory, for example, for the sake of production economy. A fusion protein containing a GLP-1 analogue or PYY₃₋₃₆ analogue backbone, as inclusion bodies, is subjected to denaturation and renaturation to obtain a fusion protein with a correct conformation. After a series of treatments such as digestion, sedimentation by adjustment, and centrifugation, the GLP-1 analogue or PYY₃₋₃₆ analogue backbone with a higher content is obtained. After further purification treatment by ion exchange chromatography, the GLP-1 analogue or PYY₃₋₃₆ analogue backbone with a higher purity is obtained.

The term “excipient” in a broad sense refers to any ingredient other than the active therapeutic ingredient. The excipient may be a pharmaceutically acceptable inert substance, inactive substance and/or not pharmaceutically active substance. Formulation of a pharmaceutically active ingredient with various excipients is known in the art. Non-limiting functional examples of excipients include, but are not limited to, a filler, a solvent, etc. Those skilled in the art can easily select excipients with suitable functions according to the dosage form of the preparation. For suitable carriers, reference can be made to [17]. The term “filler” includes, but is not limited to, lactose, sucrose, etc.; and the term “solvent” includes, but is not limited to, ethanol, water, etc.

The composition of the present disclosure can be administered pharmaceutically in a variety of preparation dosage forms (each dosage form includes immediate-release, sustained-release or controlled-slow-release dosage forms), including but not limited to tablets, capsules, injections and other dosage forms. Those skilled in the art can choose a suitable preparation dosage form according to the route of administration. The route of administration includes, but is not limited to, oral administration, percutaneous administration, subcutaneous administration, intravenous administration, intramuscular administration, etc. According to the route of administration, the dosage form is formulated on the basis of acceptable pharmaceutical operation procedures^{[18]}. All the dosage forms used are well known to relevant technicians in the pharmaceutical field, and they are administered after mixing with pharmaceutically acceptable auxiliary materials which are selected according to the selected route of administration and standard pharmaceutical operation procedures.

The composition of the present disclosure may also be administered in combination with a soluble polymer, e.g., coupling with a targeting drug carrier, cross-linking with a biodegradable polymer or hydrogel for controlling drug release, or combining with an amphiphilic block copolymer.

The term “treating a disease” refers to treating and caring for a patient who has already been afflicted with a disease, condition, or disorder. The purpose of the treatment is to resist the disease, condition, or disorder. The treatment includes the administration of an active compound to eliminate or control a disease, condition, or disorder and to alleviate symptoms or complications associated with the disease, condition or disorder.

The dosage regimen of the preparation of the present disclosure will vary depending on known factors, such as the pharmacokinetic properties of a specific agent and the mode and route of administration thereof, the species, health status, medical status, age, sex and body weight of the recipient, the nature and degree of the symptoms, the type and frequency of treatment, the kidney and liver functions of the patient, and the desired effect. Physicians or veterinarians can formulate prescriptions according to the required amount of the drug that is effective in preventing, resisting, or inhibiting the progress of the disease state.

Administration of the therapeutic agent of the present disclosure includes, but is not limited to, administering a therapeutically effective amount of the agent of the present disclosure. The term “therapeutically effective amount” refers to the amount of a therapeutic agent for treating a condition that can be treated and/or prevented by administering the composition of the present disclosure. The dosage is an amount that is sufficient to show a therapeutic or preventive or ameliorative effect. The effect includes the treatment and/or prevention of the diseases listed here. The exact and effective amount for the subject in need thereof will depend on the body weight and health status of the subject, the nature and extent of the condition to be treated, the advice given by the treating physician, and the treatment method or combination of treatment methods selected for administration. Therefore, it is useless to specify the exact and effective amount in advance.

The term “unit dose” in the present disclosure means that the composition of the present disclosure is in the form of unit dose, and these unit dose forms may be those commonly used in pharmaceutical pharmaceutics. A unit dose refers to the amount of an active pharmaceutical contained in each tablet, each capsule, and each injection.

According to general guidelines, the active ingredient will generally be present in the composition in an amount of about 0.5%-95% based on the total weight of the composition. The composition of the present disclosure can be administered in a single daily dose or in a total daily dose divided into two, three or four doses per day. The composition of the present disclosure may also be administered as a long-acting preparation, which will enable the active ingredient to be slowly released within a period of days/weeks/months as required.

### Abbreviations

| Abbreviation | Full name |
|---|---|
| BMI | Body mass index |
| ° C. | degrees centigrade |
| CKD-EPI | Chronic Kidney Disease Epidemiology Collaboration |
| CO₂ | Carbon dioxide |
| DM | Diabetes mellitus |
| DMEM culture | A culture medium containing various amino acids |
| medium | and glucose |
| EPM | Elevated plus maze |
| FL | Fatty liver |
| g | Gram |
| G1 | Referring to Formula III |
| G1P1 | Referring to Formulas III and IV in combination |
| GLP-1 | Glucagon like peptide 1 |
| GPCRs | G-protein-coupled receptors |
| GTT | Glucose tolerance test |
| h | Hour |
| HbA1c | Glycated hemoglobin A1c |
| HE staining | Hematoxylin-eosin staining |
| HFD | High-fat diet |
| IC₅₀ | Half maximal inhibitory concentration |
| ITT | Insulin tolerance test |
| IU/kg | unit per kilogram |
| KDIGO | Kidney Disease: Improving Global Outcomes |
| kg | Kilogram |
| L | Liter |
| m² | Square meter |
| mg | Milligram |
| min | Minute |
| mL or ml | Milliliter |
| mmol | Millimole |
| mol | Mole |
| ng | Nanogram |
| NS | Normal saline |
| OFT | Kuachang Shiyan |
| P1 | Referring to Formula IV |
| pH | Pondus Hydrogenii |
| PYY | Peptide tyrosine tyrosine |
| PYY₃₋₃₆ | Peptide tyrosine tyrosine 3-36 |
| SPF | Specific pathogen free |
| TST | Tail suspension test |
| μg | Microgram |
| μl or μL | Microliter |
| μmol | Micromole |

### REFERENCE

[1] Lister N B, Baur L A, Felix J F, et al. Child and adolescent obesity [J]. Nat Rev Dis Primers, 2023, 9 (1): 24.
[2] Safaei M, Sundararajan E A, Driss M, et al. A systematic literature review on obesity: Understanding the causes & consequences of obesity and reviewing various machine learning approaches used to predict obesity [J]. Computers in Biology and Medicine, 2021, 136:104754.
[3] Tomic D, Shaw J E, Magliano D J. The burden and risks of emerging complications of diabetes mellitus [J]. Nat Rev Endocrinol, 2022, 18 (9): 525-39.
[4] Antar S A, Ashour N A, Sharaky M, et al. Diabetes mellitus: Classification, mediators, and complications; A gate to identify potential targets for the development of new effective treatments [J]. Biomed Pharmacother, 2023, 168:115734.
[5] Loomba R, Friedman S L, Shulman G I. Mechanisms and disease consequences of nonalcoholic fatty liver disease [J]. Cell, 2021, 184 (10): 2537-64.
[6] Powell E E, Wong V W, Rinella M. Non-alcoholic fatty liver disease [J]. Lancet, 2021, 397 (10290): 2212-24.
[7] Drucker D J. GLP-1 physiology informs the pharmacotherapy of obesity [J]. Mol Metab, 2022, 57:101351.
[8] Drucker D J. The benefits of GLP-1 drugs beyond obesity [J]. Science, 2024, 385 (6706): 258-60.
[9] Chen W, Binbin G, Lidan S, et al. Evolution of peptide Y Y analogs for the management of type 2 diabetes and obesity [J]. Bioorg Chem, 2023, 140:106808.
[10] Samms R J, Cosgrove R, Snider B M, et al. GIPR Agonism Inhibits PYY-Induced Nausea-Like Behavior [J]. Diabetes, 2022, 71 (7): 1410-23.
[11] Boland B B, Laker R C, O'Brien S, et al. Peptide-YY (3-36)/glucagon-like peptide-1 combination treatment of obese diabetic mice improves insulin sensitivity associated with recovered pancreatic β-cell function and synergistic activation of discrete hypothalamic and brainstem neuronal circuitries [J]. Mol Metab, 2022, 55:101392.
[12] Dischinger U, Hasinger J, Königsrainer M, et al. Toward a Medical Gastric Bypass: Chronic Feeding Studies With Liraglutide+PYY (3-36) Combination Therapy in Diet-Induced Obese Rats [J]. Front Endocrinol (Lausanne), 2020, 11:598843.
[13] Gross E. The Peptides Analysis, Synthesis, Biology: Modern Techniques of Conformational Structural, and Configurational Analysis [M]. Elsevier, 2012.
[14] Schröder E, Lübke K. The Peptides: Methods of peptide synthesis [M]. Elsevier, 2014.
[15] Chan W, White P. Fmoc solid phase peptide synthesis: a practical approach [M]. OUP Oxford, 1999.
[16] Collins J M, Singh S K, White T A, et al. Total wash elimination for solid phase peptide synthesis [J]. Nat Commun, 2023, 14 (1): 8168.
[17] Paul J. S. Handbook of Pharmaceutical Excipients [M]. Pharmaceutical Press, 2020.
[18] Goodman L S. Goodman and Gilman's the pharmacological basis of therapeutics [M]. McGraw-Hill New York, 2022.

### Example 1

The example provided a composition composed of a GLP-1 polypeptide analogue and a PYY₃₋₃₆ polypeptide analogue, wherein
the amino acid sequences of the GLP-1 polypeptide analogue were as shown in Formula I:

(SEQ ID NO: 3)
HX₂ₐX₃ₐ GTF TSD VSS YLE GQA AKX₂₁ₐ FIA WLX₂₇ₐ KX₂₉ₐR X₃₁ₐ
Formula I

X₂ₐ was selected from the group consisting of valine, isoleucine, glycine, threonine, leucine, and serine;
X₃ₐ was selected from the group consisting of serine and alanine;
X₂₁ₐ was selected from the group consisting of arginine and lysine;
X₂₇ₐ was selected from the group consisting of serine and alanine;
X₂₉ₐ was selected from the group consisting of alanine, serine, threonine, and methionine; and
X₃₁ₐ was selected from the group consisting of alanine, serine, threonine, and methionine; and
the amino acid sequences of the PYY₃₋₃₆ analogue were as shown in Formula II:

(SEQ ID NO: 4)
IKP EAP GEX_{11b} ASP EEL X_{18b}X_{19b}Y YAX_{23b} LRH X_{27b}LN X_{30b}VT RQR Y
Formula II

X_{11b} was selected from the group consisting of aspartic acid, glycine, glutamic acid, and asparagine;
X_{18b} was selected from the group consisting of aspartic acid, glutamine, and histidine;
X_{19b} was selected from the group consisting of arginine and lysine;
X_{23b} was selected from the group consisting of serine, threonine, alanine, glycine, and methionine;
X_{27b} was selected from the group consisting of tyrosine, tryptophan, and phenylalanine; and
X_{30b} was selected from the group consisting of leucine, alanine, isoleucine, valine, and methionine.

Without changing the secondary structure of Formula I or II, amino acid substitutions at a specific site were realized according to the principle of conservative substitutions of amino acids.

### Example 2

The example provided a composition composed of a GLP-1 polypeptide analogue and a PYY₃₋₃₆ polypeptide analogue, wherein
the amino acid sequences of the GLP-1 polypeptide analogue were as shown in Formula III:

(SEQ ID NO: 1)
HGE GTF TSD VSS YLE GQA AKE FIA WLV KSRA
Formula III.

The amino acid sequences of the PYY₃₋₃₆ analogue were as shown in Formula IV:

(SEQ ID NO: 2)
IKP EAP GED ASP EEL VRY YAG LRH WLN LVT RQRY
Formula IV.

### Example 3: Cytotoxicity of Formula III and Formula IV Administered in Combination

Mixture solutions of Formulas III and IV with gradient molar concentrations were respectively prepared with a high-sugar DMEM complete medium (Wuhan Servicebio Technology Co., Ltd., the same below). The gradient molar concentrations of each of the mixture solutions of Formulas III and IV were 2 μmol/mL (meaning that the concentrations of Formulas III and IV were both 2 μmol/mL, the same below), 1 μmol/mL, 0.5 μmol/mL, 0.25 μmol/mL, and 0.125 μmol/mL, respectively. HEK-293t cells (5000 cells/100 μL/well) were added to a 96-well plate (37±0.5° C., 5±0.1% CO₂) and cultured for 24 h, and 100 μL of the mixture solutions of Formulas III and IV with gradient molar concentrations were then separately added to the 96-well plate. Detection was carried out using a CCK-8 kit (Wuhan Servicebio Technology Co., Ltd.). The absorbance at 450 nm was measured by a microplate reader, and the cell survival rate was calculated. An IC₅₀ curve was plotted by GraphPad Prism.

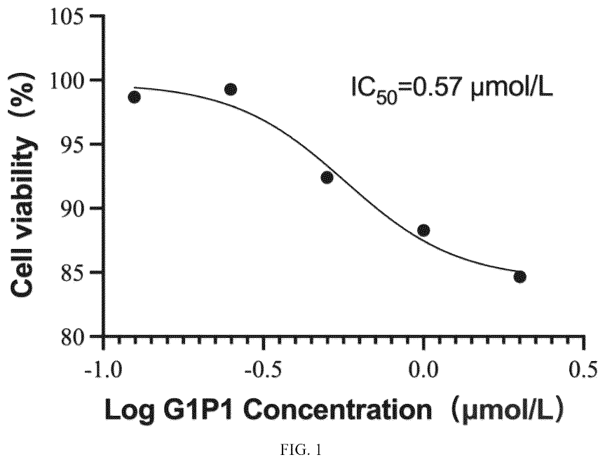

The results were as shown in FIG. 1.

When Formulas III and IV were used in equal proportion, the IC₅₀ was 0.57 μmol/L.

### Example 4: Effects of Formula III and Formula IV Administered in Combination on Body Weight of Mice

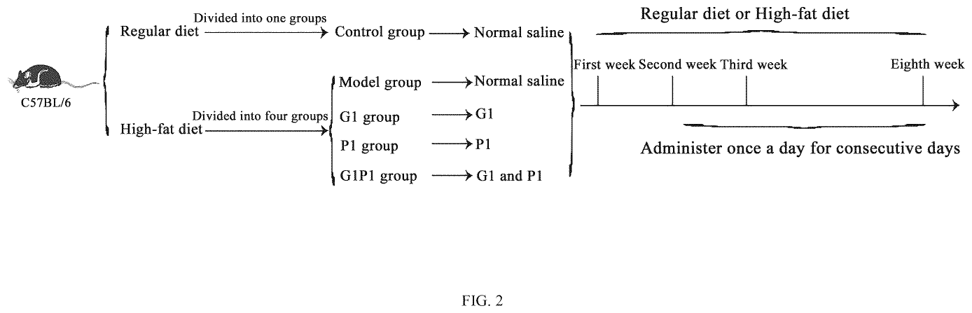

Fifty SPF male C57BL/6 mice (SiPeiFu (Beijing) Biotechnology Co., Ltd., the same below) were raised in an environment at a temperature of 22° C. to 26° C. and a relative humidity of 40%-70%, with ventilation at 8-12 times/h. The mice were randomly divided into five groups (10 mice/group, as shown in FIG. 2), i.e., a control group (denoted by Control, the same below), a Model group (denoted by Model, the same below), a Formula III group (denoted by G1 group herein and by G1, the same below), a Formula IV group (denoted by P1 group herein and by P1, the same below), and a Formulas III+IV combination group (denoted by G1P1 group herein and by G1P1, the same below), respectively.

The model group, G1 group, P1 group, and G1P1 group were given 60%-80% high-fat feed (HFD) on days 1-7 for adaptive diet induction and 100% high-fat feed on days 8-55. On days 15-55, the model group was injected with 0.9% normal saline (NS), the G1 group was injected with 60 μmol/kg of Formula III, the P1 group was injected with 80 μmol/kg of Formula IV, and the G1P1 group was injected with 60 μmol/kg of Formula III and 80 μmol/kg of Formula IV, on a daily basis by subcutaneous injection, for 40 days. The C57BL/6 mice were administered in a fasted state. The daily weight change and the total food intake 48 h after each administration were measured.

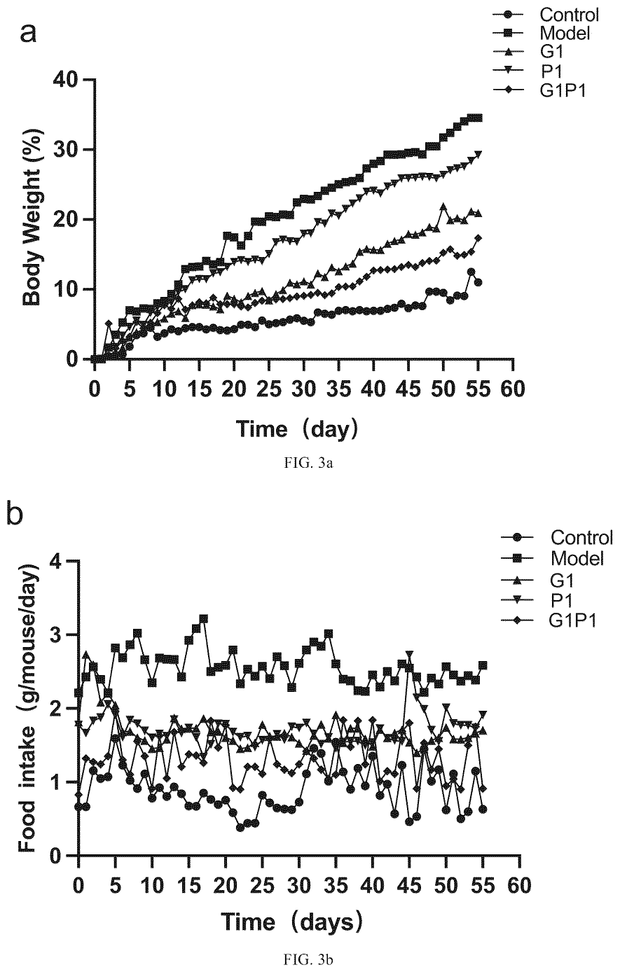

The detection results were as shown in FIGS. 3a and 3b.

Starting on day 15, the body weights of the five groups of mice became significantly different. Compared with the control group, the body weight in the model group increased significantly by 40%-70%. Compared with the model group, the body weights in the G1 group, P1 group, and G1P1 group decreased by about 5%-45%, 5%-30%, and 5%-55%, respectively. Given the same high-fat diet, compared with the G1 group and P1 group, the body weight in the G1P1 group decreased by about 5%-20% and 5%-45%, respectively.

Compared with the control group, the food intake in the model group increased significantly by 30%-75%. Compared with the model group, the food intakes in the G1 group, P1 group, and G1P1 group decreased by about 5%-50%, 5%-45%, and 5%-60%, respectively; and compared with the G1 group and P1 group, the food intake in the G1P1 group decreased by about 5%-60% and 5%-45%, respectively.

### Example 5: Effect of Combined Administration of G1P1 on Anxiety and Depression

The mice in each group in Example 4 were subjected to a behavioral test.

Open field test (OFT): The mice were put into a test box to detect their activities during the test time. Elevated plus maze (EPM): The mice were placed in the center of an elevated plus maze to observe their activities during the test time. Tail suspension test (TST): The tails of the mice were hung on a cage rack, and the behavior patterns of struggle, swing and rest were observed during suspension.

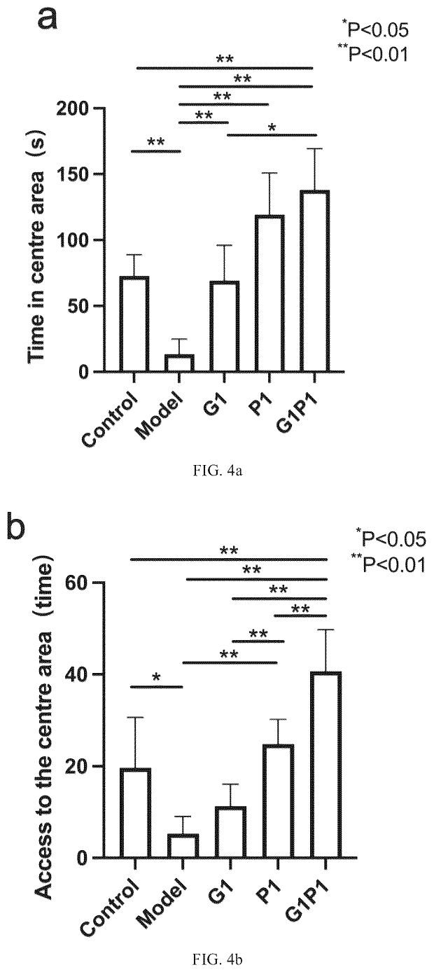

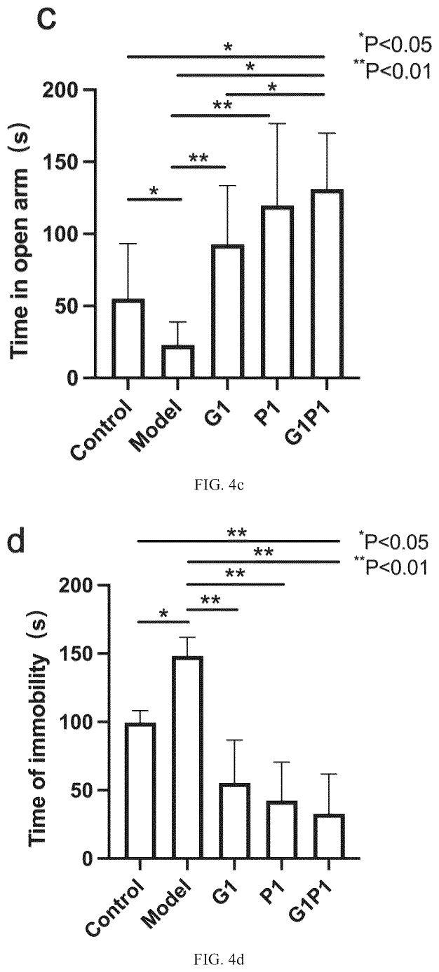

The results were as shown in FIGS. 4a-4d.

In OFT and EPM tests, compared with the model group, the mice in the control group, G1 group, P1 group, and G1P1 group had a significantly increased entry time in the central area and a significantly increased number of accesses to the central area, and a significantly increased entry time in the open arm area. Compared with the control group, G1 group, and P1 group, the G1P1 group had an increased entry time in the central area and an increased number of accesses to the central area and an increased entry time in the open arm area.

In the TST test, compared with the mice in the control group, G1 group, P1 group, and G1P1 group, the mice in the model group had an increased immobility time, and compared with the control group, G1 group, and P1 group, the G1P1 group had a decreased immobility time.

The above results showed that obesity could aggravate the anxiety and depression symptoms of the mice, and the combined administration of G1P1 to the mice with anxiety and depression symptoms could significantly ameliorate or even prevent the anxiety and depression symptoms of the mice as compared with the administration of G1 or P1 alone. The results of comparison between the G1P1 group and the control group showed that the combined administration of G1P1 could effectively prevent normal mice from suffering from anxiety and depression.

### Example 6: Effect of Combined Administration of G1P1 on Glucose Tolerance

The steps of the glucose tolerance test (GTT) were as follows:

This test was carried out after the mice in Example 5 had recovered. Before the test, the mice were fasted for one day, during which they drank water normally. An aqueous solution with a glucose content of 20% was given by gavage, with the gavage volume being 10 μL/g (administered according to body weight). The blood glucose value was measured with a blood glucose meter at 15 min, 30 min, 60 min, 90 min, and 120 min.

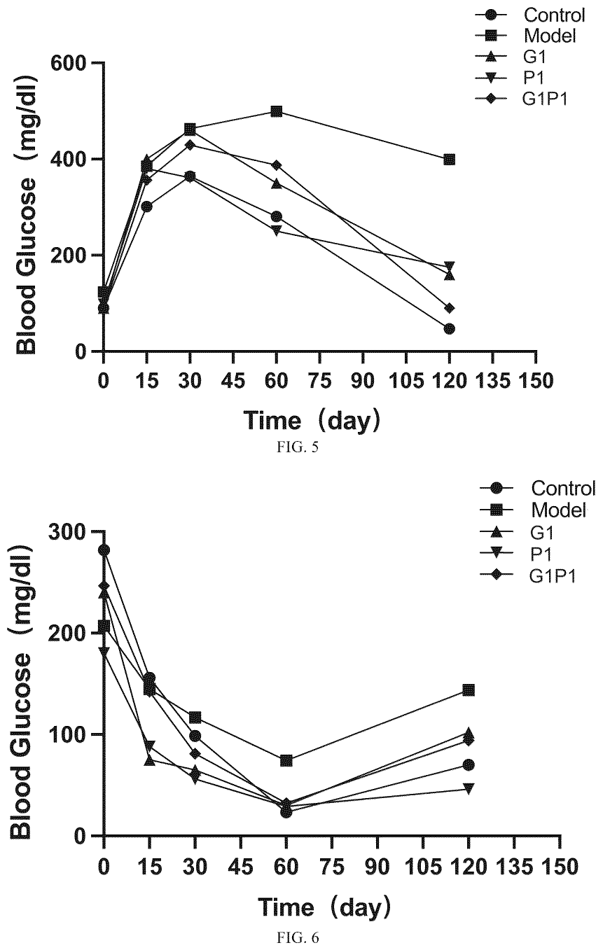

The results of the test with the blood glucose meter were as shown in FIG. 5.

Compared with the control group, G1 group, P1 group, and G1P1 group, the blood glucose in the model group within 0-120 min increased by about 5%-95%, 5%-60%, 5%-55%, and 5%-80%; and the blood glucose in the G1P1 group within 0-120 min decreased by about 5%-35% than G1 and decreased by about 10%-45% than the P1 group, indicating that the combined administration of G1P1 was more effective in lowering blood glucose than the single drug treatments.

### Example 7: Effect of Combined Administration of G1P1 on Insulin Glucose Tolerance

The steps of the insulin tolerance test (ITT) were as follows:

After the completion of Example 6, this test was carried out after the mice had recovered for 3 days. Before the test, the mice were fasted for one day, during which they drank water normally. Each mouse was intraperitoneally injected with an insulin solution at 0.75 IU/kg, and the blood glucose value was determined using a blood glucose meter and blood glucose test paper at 15 min, 30 min, 60 min, 90 min, and 120 min.

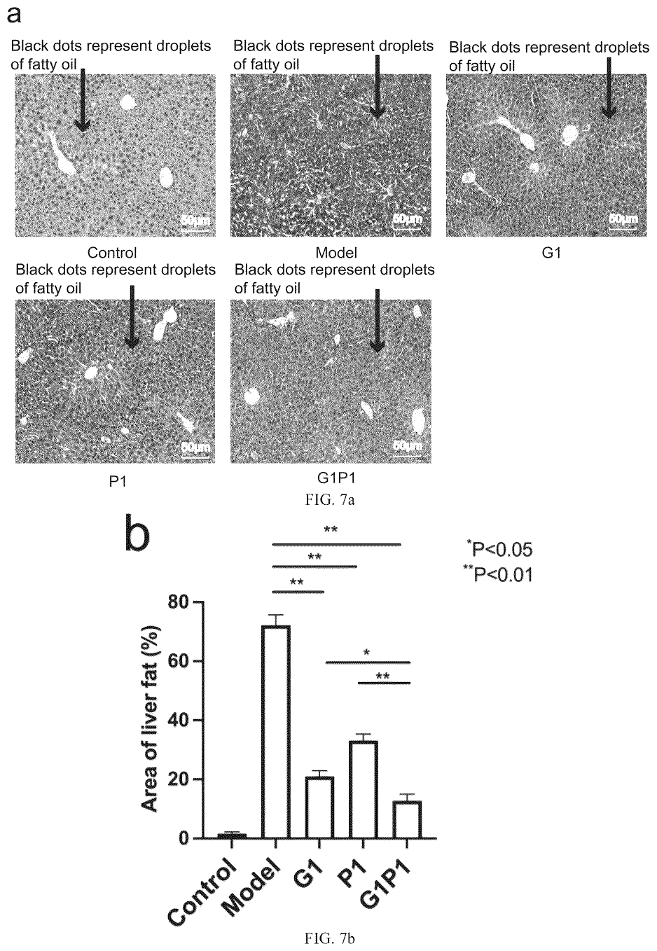

The results of the test with the blood glucose meter were as shown in FIG. 6.

Within 0-120 min, the blood glucose decrease in each group was about 5%-85% for the control group, about 5%-30% for the model group, about 5%-55% for the G1 group, about 5%-65% for the P1 group, and about 10%-75% for the G1P1 group. Compared with the model group, the G1 group, P1 group, and G1P1 group had improved insulin tolerance 2 h after intraperitoneal insulin injection, and as compared with the G1 group and P1 group, the blood glucose in the G1P1 group decreased by about 5%-20% and 5%-10%, respectively, indicating that the combined administration of G1P1 was more effective in insulin tolerance than the single drugs.

### Example 8: Effect of Combined Administration of G1P1 on Body Fat Accumulation

After the completion of Example 7, the mice were directly anesthetized and then fixed on a rat plate supinely, the outer skin was dissected to expose subcutaneous fat, and the subcutaneous fat accumulation was recorded. Subsequently, organs were exposed, and the inter-organ fat accumulation was observed. Finally, leg muscle and liver tissues were collected from the mice and stored in a refrigerator at −80° C.

The subcutaneous and inter-organ fat accumulation of the mice in the model group increased by about 35%-65% as compared with the control group, and the subcutaneous and inter-organ fat accumulation in the G1P1 group increased by 10%-20% as compared with the control group, decreased by about 5%-30% as compared with the model group, and decreased by about 5%-10% as compared with the G1 group and the P1 group. This demonstrated that the G1P1 group had a remarkable control effect in reducing subcutaneous and inter-organ fat accumulation, and the combined administration of G1P1 was more effective in controlling body fat accumulation than the single drugs.

### Example 9: Effect of Combined Administration of G1P1 on Fatty Liver

The mouse liver tissue collected in Example 8 was cryosectioned, and the liver tissue sections were fixed with formaldehyde-calcium, then washed, and soaked in 60% isopropanol. A saturated oil red O stock solution (Beijing Solarbio Science & Technology Co., Ltd.) was diluted at 3:2 (oil red O: distilled water) for staining, and the sections were mounted with glycerol gelatin and examined under a microscope.

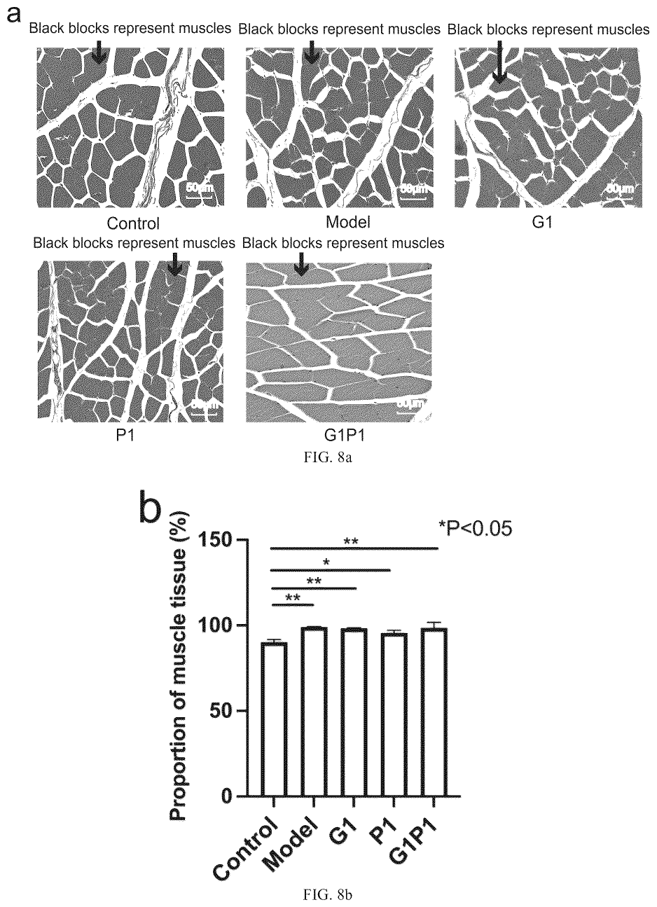

The results were as shown in FIGS. 7a and 7b.

In terms of oil red O staining of the mouse liver tissue, the content of fatty oil droplets in the model group significantly increased by about 70%-99% as compared with the control group; the contents of fatty oil droplets in the G1 group, P1 group, and G1P1 group significantly decreased by about 40%-65%, 40%-55%, and 50%-85% as compared with the model group; and the content of fatty oil droplets in the G1P1 group decreased by about 10%-45% and 10%-60% as compared with the G1 group and P1 group, respectively. This demonstrated that the combined administration of G1P1 had a remarkable effect in reducing liver fat accumulation and was more effective in treating fatty liver than the single drugs.

### Example 10: Effect of Combined Administration of G1P1 on Muscle

The mouse muscle tissue collected in Example 8 was cryosectioned. After staining with a hematoxylin solution and an eosin solution (Beijing Solarbio Science & Technology Co., Ltd.), the sections were mounted with glycerol gelatin and examined under a microscope.

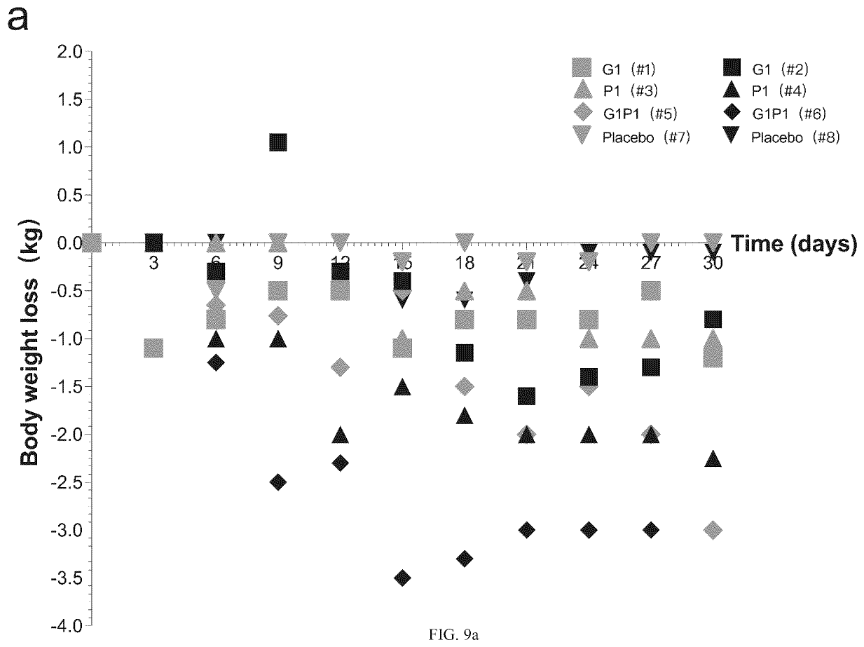

The results were as shown in FIGS. 8a and 8b.

Compared with the mice in the control group, the muscle areas in the mice of the model group, G1 group, P1 group, and G1P1 group increased by 5%-20%, 5%-15%, 5%-10%, and 5%-20%, and compared with the model group, the muscle areas in the mice of the G1 group, P1 group, and G1P1 group had no significant difference.

### Example 11: Research-Oriented Human Test of Combined Administration of G1P1

With the informed consent, qualified volunteers (with no adverse reactions found before administration) were recruited, and compared with Placebo, the administration of G1 or P1 alone and the combined administration of G1P1 showed therapeutic effectiveness in body weight control in an overweight or obese population.

Blind method: single blind. Eight volunteers were randomly divided into four groups, which were respectively given capsules containing corresponding drugs or a placebo (made by Gene Young Biopharma Ltd. (Shenzhen)) once a day, one capsule at a time, with the course of administration being 30 days. The body weight was measured before administration. The administration was carried out in 50-100 mL of drinking water between 1.5 h and 2 h after breakfast each day, with the dosing timing being between 09:45 and 10:15 AM. Within 30 minutes after administration, substantial intake of water and food was not allowed; thereafter, normal diet and drinking were both resumed.

The inclusion criteria of volunteers were as shown in Table 1.

TABLE 1
Inclusion criteria of volunteers

| | |
|---|---|
| Inclusion | 1. The informed consent was obtained before any research-related activities. |
| criteria | Research-related activity refers to any procedures implemented as part of the |
| | research, including determining activities suitable for inclusion in the research. |
| | 2. Male or female, aged ≥18 at the time of signing the informed consent. |
| | 3. Body mass index (BMI) ≥24.0 kg/m² |
| | 4. Self-reported unsuccessful diet weight loss history at least once. |
| | 5. The body weight was controlled only by diet and exercise and was stable for at |
| | least 30 days before the screening day. |
| Exclusion | 1. Subjects who did not have type II diabetes mellitus at the time of screening: |
| criteria | however, HbA1c ≥6.5% (48 mmol/mol) as measured by a central laboratory at the |
| | time of screening. |
| | 2. Had a history of type I and type II diabetes mellitus. |
| | 3. Had been treated with glucose-lowering drugs within 90 days before screening. |
| | 4. Those who drank alcohol frequently within 3 months before the test, i.e., those |
| | who consumed more than 2 units of alcohol on average per day ((1 unit = 360 mL |
| | of beer, or 45 mL of a spirit with an alcohol content of 40%, or 150 L of grape wine), |
| | or those whose alcohol breath test result was positive at the time of screening. |
| | 5. Those who had participated in other clinical tests within 3 months before the test. |
| | 6. Those who had a history of taking psychoactive substances within 3 months |
| | before the test, or had a history of drug abuse within 12 months before the test, or |
| | had positive urine drug screening results. |
| | 7. Pregnant or lactating females, or females who were ready to conceive within 3 |
| | months, or males who were ready to father a child within 3 months. |
| | 8. Those with special dietary requirements. |
| | 9. Those with renal dysfunction after screening, i.e., with a glomerular filtration |
| | rate <30 mL/min/1.73 m² as calculated using a CKD-EPI creatinine equation defined |
| | by KDIGO 2012 classification, as estimated by the central laboratory. |
| | 10. Other subjects judged by the researcher as unsuitable for the study. |

The grouping in the test was as shown in Table 2.

TABLE 2
Grouping for human test

| | | | Initial | | | | |
|---|---|---|---|---|---|---|---|
| | | | body | | BMI/ | | Drug |
| No. | Gender | Age | weight/kg | Height/cm | (kg/m²) | Administration | specification |
| #1 | Female | 53 | 73.6 | 166 | 26.9 | G1 group | G1: 14 mg |
| #2 | Male | 29 | 91.8 | 178 | 28.4 | G1 group | G1: 14 mg |
| #3 | Female | 23 | 64 | 159 | 25.3 | P1 group | P1: 14 mg |
| #4 | Male | 50 | 72 | 168 | 25.5 | P1 group | P1: 14 mg |
| #5 | Female | 59 | 89 | 171 | 30.1 | G1P1 group | G1: 14 mg |
| | | | | | | | P1: 14 mg |
| #6 | Male | 50 | 117.5 | 180 | 36.3 | G1P1 group | G1: 14 mg |
| | | | | | | | P1: 14 mg |
| #7 | Female | 45 | 62 | 160 | 24.6 | Placebo group | Placebo: 14 |
| | | | | | | | mg |
| #8 | Male | 55 | 64 | 163 | 24.1 | Placebo group | Placebo: 14 |
| | | | | | | | mg |

The evaluation criteria were as shown in Table 3.

TABLE 3
Evaluation criteria for human test

| | | | | Selection of |
|---|---|---|---|---|
| | | | | endpoint |
| | No. | Indicator | Evaluation time | indicator |
| Primary | 1 | Relative | From baseline (day 0) | Effectiveness |
| endpoint | | change in | to the end of treatment | indicator |
| indicators | | body weight | (Day 30) | |
| and | 2 | Body weight | At the end of the | Effectiveness |
| evaluation | | loss ≥1% | treatment | indicator |
| time | | (Yes/No) | (Day 30) | |
| Secondary | 1 | Body weight | At the end of the | Effectiveness |
| endpoint | | loss ≥5% | treatment | indicator |
| indicators | | (Yes/No) | (Day 30) | |
| and | 2 | Body weight | At the end of the | Effectiveness |
| evaluation | | loss ≥10% | treatment | indicator |
| time | | (Yes/No) | (Day 30) | |
| | 3 | Body weight | At the end of the | Effectiveness |
| | | loss ≥15% | treatment | indicator |
| | | (Yes/No) | (Day 30) | |
| | 4 | Change in BMI | From baseline (day 0) | Effectiveness |
| | | | to the end of treatment | indicator |
| | | | (Day 30) | |

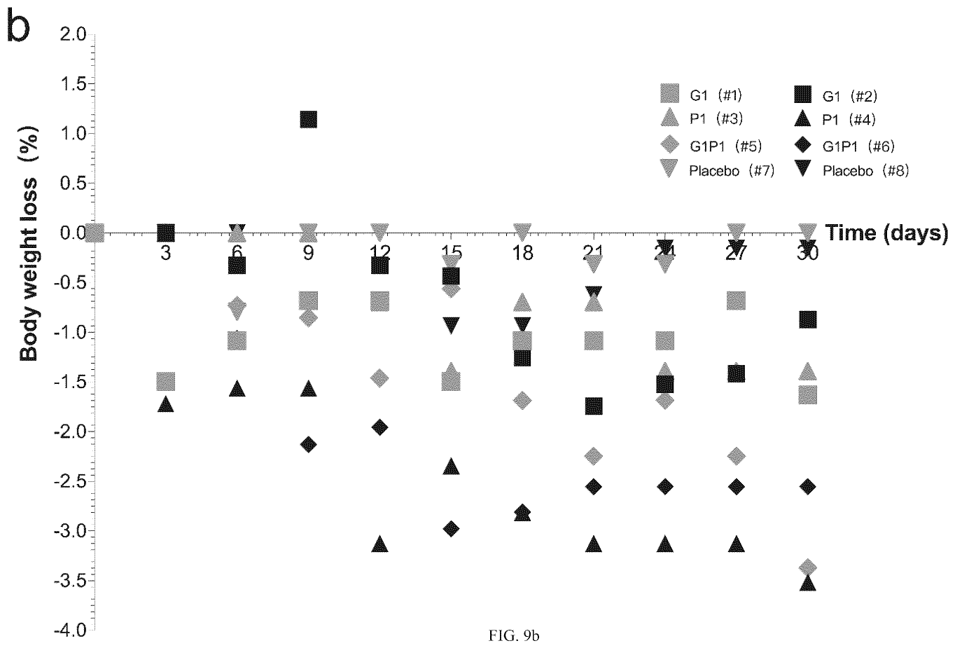

The results were as shown in Table 4 and FIGS. 9a and 9b.

TABLE 4
Statistical table of body weight data during human test

| | Body weight (kg) | | | | | | | |
|---|---|---|---|---|---|---|---|---|
| Time (day) | #1 | #2 | #3 | #4 | #5 | #6 | #7 | #8 |
| 0 | 73.6 | 91.8 | 64 | 72 | 89 | 117.5 | 62 | 64 |
| 3 | 73.6 | 90.7 | 62.9 | 72 | 89 | 117.5 | 62 | 64 |
| 6 | 73.3 | 91 | 63 | 72 | 88.35 | 116.25 | 61.5 | 64 |
| 9 | 74.65 | 91.3 | 63 | 72 | 88.24 | 115 | 62 | 64 |
| 12 | 73.3 | 91.3 | 62 | 71.5 | 87.7 | 115.2 | 62 | 64 |
| 15 | 73.2 | 90.7 | 62.5 | 71 | 88.5 | 114 | 61.8 | 63.4 |
| 18 | 72.45 | 91 | 62.2 | 71.5 | 87.5 | 114.2 | 62 | 63.4 |
| 21 | 72 | 91 | 62 | 71.5 | 87 | 114.5 | 61.8 | 63.6 |
| 24 | 72.2 | 91 | 62 | 71 | 87.5 | 114.5 | 61.8 | 63.9 |
| 27 | 72.3 | 91.3 | 62 | 71 | 87 | 114.5 | 62 | 63.9 |
| 30 | 72.8 | 90.6 | 61.75 | 71 | 86 | 114.5 | 62 | 63.9 |

From the perspective of gender, in terms of effectiveness, the males in the G1, P1 and G1P1 dosage groups showed body weight losses of about 0.1 kg-1.2 kg, 0.1 kg-1.0 kg, and 0.1 kg-3.5 kg over days 0-30, and the weight loss percentages were 0.1%-1.3%, 0.1%-1.4%, and 0.1%-3%, respectively; and the females in the G1, P1 and G1P1 dosage groups showed body weight losses of about 0.1 kg-1.6 kg, 0.1 kg-2.3 kg, and 0.1 kg-3.0 kg, and the weight loss percentages were 0.1%-2.2%, 0.2%-3.6%, and 0.5%-3.4%, respectively. On the basis of similar BMIs, in terms of body weight control, different modes of administration (the administration of the G1P1 combination and G1 and P1 alone) demonstrated a superior body weight control effect in females to that in males. Furthermore, the administration of the G1P1 combination showed a superior effect to G1 or P1 alone.

From the perspective of BMI, in terms of effectiveness, in days 0-30, in the case of BMI=24-28 kg/m², the G1 and P1 dosage groups showed body weight losses of about 0.1 kg-1.4 kg and 0.1 kg-1.7 kg, and the weight loss percentages were 0.1%-1.7% and 0.5%-2.5%, respectively. In the case of BMI>28 kg/m², the G1P1 dosage group showed a weight loss of about 0.3 kg-3.5 kg, and the weight loss percentage was 0.2%-3.4%. For the administration of the G1P1 combination and G1 and P1 alone, in terms of body weight control, the higher the BMI, the more significant the body weight control effect, and the administration of the G1P1 combination had a superior effect to the administration of G1 or P1 alone.

In summary, in terms of effectiveness, the administration of G1, P1, and the G1P1 combination caused a body weight boss of at least 1% in the 30-day dosage period, which reached the expectation. The higher the BMI, the better the body weight control effect. In the case of similar BMIs, the body weight control effect in females was superior to that in males. Furthermore, the administration of the G1P1 combination showed a superior effectiveness to the administration of G1 or P1 alone.

### Example 12: Preparation of Combined G1P1 Preparation

### A) Tablets:

Tablets could be prepared according to conventional tablet preparation operating procedures, whereby tablets with the preparation specifications were obtained. The tablets were made up of, for example, 14 g of Formula III, 14 g of Formula IV, 0.02 g of colloidal silicon dioxide, 0.5 g of magnesium stearate, 27.5 g of microcrystalline cellulose, 1.1 g of starch, and 9.88 g of lactose. An appropriate coating could be used to improve the taste or delay absorption. 1000 tablets were be prepared.

### B) Capsules:

Capsules can be prepared according to conventional capsule preparation procedures, so as to obtain capsules with the preparation specifications. For example, 1000 capsules were prepared by filling standard hard gelatin capsules with 14 g of Formula III, 14 g of Formula IV, 15 g of lactose, 5 g of microcrystalline cellulose, 0.6 g of magnesium stearate, and an appropriate amount of an absorbent promoter.

### C) Injection:

An injection could be prepared by conventional steps. For example, an injection preparation with the following composition was prepared by the method described below.

5 g of Formula III and 5 g of Formula IV were dissolved in a portion of water for injection, and 40 g of sulfobutyl cyclodextrin was added to the drug solution and stirred for about 8 min. Sodium hydroxide or hydrogen chloride was added to adjust the pH value between 6 and 8. Water for injection and a sodium chloride aqueous solution were added to achieve a final volume of 1 L and make the solution isotonic. The solution was then divided and filled into appropriate ampoules to prepare 1000 vials of injections. Before adjusting the pH value, other inactive components such as solvents, cosolvents, solubilizers, emulsifiers or thickeners, chelating agents, antioxidants, reducing agents, antimicrobial preservatives, buffers, expansion agents, protective agents, tension regulators, and special additives could be added as required.

The injection preparation was necessarily sterile, pyrogen-free, and free of particulate matters when it was a solution. It can be prepared by stirring in a pharmaceutically acceptable buffer which may or may not contain a cosolvent or other excipients. Before use, the solution should be made isotonic with pharmaceutical sodium chloride and sterilized.

The examples of the present disclosure have been described in detail above in conjunction with the examples, but the present disclosure is not limited to the above examples. Various changes can also be made within the knowledge of those of ordinary skill in the art to which the present disclosure belongs without departing from the gist of the present disclosure.

What is claimed:

1. A composition comprising a GLP-1 polypeptide analogue and a PYY₃₋₃₆ polypeptide analogue,
wherein amino acid sequences of the GLP-1 polypeptide analogue are as shown in Formula I:
(SEQ ID NO: 3)
HX₂ₐX₃ₐ GTF TSD VSS YLE GQA AKX₂₁ₐ FIA WLX₂₇ₐ KX₂₉ₐR X₃₁ₐ
Formula I
X₂ₐ is selected from the group consisting of valine, isoleucine, glycine, threonine, leucine, and serine;
X₃ₐ is selected from the group consisting of serine and alanine;
X₂₁ₐ is selected from the group consisting of arginine and lysine;
X₂₇ₐ is selected from the group consisting of serine and alanine;
X₂₉ₐ is selected from the group consisting of alanine, serine, threonine, and methionine; and
X₃₁ₐ is selected from the group consisting of alanine, serine, threonine, and methionine;
wherein amino acid sequences of the PYY₃₋₃₆ polypeptide analogue are as shown in Formula II:
(SEQ ID NO: 4)
IKP EAP GEX_{11b} ASP EEL X_{18b}X_{19b}Y YAX_{23b} LRH X_{27b}LN X_{30b}VT RQR Y
Formula II
X_{11b} is selected from the group consisting of aspartic acid, glycine, glutamic acid, and asparagine;
X_{18b} is selected from the group consisting of aspartic acid, glutamine, and histidine;
X_{19b} is selected from the group consisting of arginine and lysine;
X_{23b} is selected from the group consisting of serine, threonine, alanine, glycine, and methionine;
X_{27b} is selected from the group consisting of tyrosine, tryptophan, and phenylalanine; and
X_{30b} is selected from the group consisting of leucine, alanine, isoleucine, valine, and methionine.

2. The composition according to claim 1, wherein the amino acid sequence of the GLP-1 polypeptide analogue is as shown in Formula III:
(SEQ ID NO: 1)
HGE GTF TSD VSS YLE GQA AKE FIA WLV KSRA
Formula III.

3. The composition according to claim 1, wherein the amino acid sequence of the PYY₃₋₃₆ polypeptide analogue is as shown in Formula IV:
(SEQ ID NO: 2)
IKP EAP GED ASP EEL VRY YAG LRH WLN LVT RQRY
Formula IV.

4. The composition according to claim 1, wherein the mass ratio of the GLP-1 polypeptide analogue to the PYY₃₋₃₆ polypeptide analogue is 1:0.25-4.

5. A method for reducing food intake, comprising administering a therapeutically effective amount of the composition according to claim 1 to a subject in need thereof.

6. A method for reducing body weight, comprising administering a therapeutically effective amount of the composition according to claim 1 to a subject in need thereof.

7. A method for increasing insulin sensitivity, comprising administering a therapeutically effective amount of the composition according to claim 1 to a subject in need thereof.

8. A method for inhibiting fat accumulation, comprising administering a therapeutically effective amount of the composition according to claim 1 to a subject in need thereof.

9. The method according to claim 8, wherein the fat accumulation includes at least one of subcutaneous fat accumulation, inter-organ fat accumulation, and liver fat accumulation.

10. A method for preventing and/or treating a disease, comprising administering a therapeutically effective amount of the composition according to claim 1 to a subject in need thereof.

11. The method according to claim 10, wherein the disease includes at least one of obesity and related complications, diabetes mellitus and related complications, glucose intolerance and related diseases caused by glucose intolerance, hyperglycemia, hyperinsulinemia, hypertension, dyslipidemia, cognitive disorder, atherosclerosis, myocardial infarction, cardiovascular disease, stroke, related diseases caused by intestinal permeability disorder, apoplexy and/or hemorrhagic apoplexy, neuroinflammation, rheumatic or rheumatoid arthritis, nephropathy, asthma, chronic obstructive pulmonary disease, dysmetabolic syndrome, mood disorder, neurodegenerative disease, and systemic lupus erythematosus.

12. The method according to claim 11, wherein the related diseases caused by intestinal permeability disorder include at least one of inflammatory bowel syndrome, dyspepsia, ulcerative colitis, and Crohn's disease.

13. The method according to claim 11, wherein the mood disorder includes at least one of anxiety and depression.

14. The method according to claim 11, wherein the neurodegenerative disease includes Alzheimer's disease.

15. A drug comprising the composition according to claim 1 and an optional pharmaceutically acceptable excipient.

16. The drug according to claim 15, wherein a dosage form of the drug is selected from the group consisting of tablets, capsules, and an injection.

17. A health care product comprising the composition according to claim 1.

18. A nutritional supplement comprising the composition according to claim 1.

19. A food additive ingredient comprising the composition according to claim 1.


SEQUENCE LISTING (4 sequences)

SEQ ID NO: 1 - protein, 31 aa, synthetic construct
HGEGTFTSDVSSYLEGQAAKEFIAWLVKSRA

SEQ ID NO: 2 - protein, 34 aa, synthetic construct
IKPEAPGEDASPEELVRYYAGLRHWLNLVTRQRY

SEQ ID NO: 3 - protein, 37 aa, synthetic construct
  VAR_SEQ 2: note = valine, isoleucine, glycine, threonine, leucine, or serine
  VAR_SEQ 3: note = serine or alanine
  VAR_SEQ 21: note = arginine or lysine
  VAR_SEQ 27: note = serine or alanine
  VAR_SEQ 29: note = alanine, serine, threonine, or methionine
  VAR_SEQ 31: note = alanine, serine, threonine, or methionine
HXAXAGTFTSDVSSYLEGQAAKXAFIAWLXAKXARXA

SEQ ID NO: 4 - protein, 40 aa, synthetic construct
  VAR_SEQ 11: note = aspartic acid, glycine, glutamic acid, or asparagine
  VAR_SEQ 18: note = aspartic acid, glutamine, or histidine
  VAR_SEQ 19: note = arginine or lysine
  VAR_SEQ 23: note = serine, threonine, alanine, glycine, or methionine
  VAR_SEQ 27: note = tyrosine, tryptophan, or phenylalanine
  VAR_SEQ 30: note = leucine, alanine, isoleucine, valine, or methionine
IKPEAPGEXBASPEELXBXBYYAXBLRHXBLNXBVTRQRY